(19) United States
(12) Patent Application Publication
(10) Pub. No.: US 2014/0128792 A1
(43) Pub. Date: May 8, 2014

(54) HEMODIALYSIS ACCESS PORT AND CLEANING SYSTEM

(71) Applicant: The Johns Hopkins University, Baltimore, MD (US)

(72) Inventors: Yuan-Ping Li, Duluth, GA (US); Sherri Jane Hall, Baltimore, MD (US); Kelvin Hong, Woodstock, MD (US); Shishira Nagesh, Baltimore, MD (US); Mary Katherine O'Grady, Baltimore, MD (US); Thomas Reifsnyder, Baltimore, MD (US); Thora Thorgilsdottir, Baltimore, MD (US); Clifford R. Weiss, Annapolis, MD (US); Brandon Doan, Peoria, AZ (US); Soumyadipta Acharya, Baltimore, MD (US)

(73) Assignee: THE JOHNS HOPKINS UNIVERSITY, Baltimore, MD (US)

(21) Appl. No.: 14/069,537

(22) Filed: Nov. 1, 2013

Related U.S. Application Data
(63) Continuation of application No. 13/465,090, filed on May 7, 2012, now abandoned.
(60) Provisional application No. 61/599,474, filed on Feb. 16, 2012.

(51) Int. Cl. A61M 1/36

(52) U.S. Cl. CPC A61M 1/3653
USPC 604/6.16; 604/244

(57) ABSTRACT

An embodiment in accordance with the present invention provides a device and method for hemodialysis including a needle access port having a housing defining a septum, wherein the septum is configured for needle access. Generally, the needle access port will have three septa for communication with valves configured to be anastomosed to a large-diameter blood vessel. The valves are in fluid communication with a flow of blood through the large-diameter blood vessel. The device also includes an elongate tube having a first and a second elongate lumen, wherein a first end of the tube is coupled to the needle access port and a second end of the tube is coupled to the valve. The device can be cleaned by injecting a cleaning fluid into a first septum of the needle access port and extracting it though the second septum of the needle access port.

### CROSS REFERENCE TO RELATED APPLICATIONS

This application is a Continuation of U.S. patent application Ser. No. 13/465,090 filed May 7, 2012 which claims the benefit of U.S. Provisional Patent Application No. 61/599,474, filed Feb. 16, 2012, the content of each of the aforementioned applications is herein incorporated by reference in their entirety.

### FIELD OF THE INVENTION

The present invention relates generally to renal therapy. More particularly, the present invention relates to a device for facilitating hemodialysis.

### BACKGROUND OF THE INVENTION

Approximately 300,000 patients undergo hemodialysis in the United States alone, with approximately 100,000 new hemodialysis patients being added each year. In order to perform hemodialysis, vascular access to the patient's blood stream is required. Currently, the options for permanent hemodialysis access are fistulas and grafts, which generally speaking are abnormal connections made between a peripheral artery and vein. Such connections essentially bypass the capillary system, thereby providing the larger flow rates required for hemodialysis. However, the life span of grafts and fistulas are low: 3 to 4 years for fistulas and 1.5 years for grafts.

Also, these connections require many interventions during their lifespan, which not only increases morbidity, but also adds large costs to the healthcare system. The failure of these connections is related to the development of stenosis due to constant large flow rates. After, repeated surgeries an access site will fail and a new access site is used until no access sites are left. A lack of remaining access sites accounts for 18% of hemodialysis patient mortality. In addition, innovation related to vascular access for hemodialysis has focused on incremental improvements to grafts and catheters, with little to no development of alternative methods and devices.

It would therefore be advantageous to provide a device and method for hemodialysis that does not require the connection of an artery and vein using a graft or fistula.

### SUMMARY OF THE INVENTION

The foregoing needs are met, to a great extent, by the present invention, wherein in one aspect, a device for hemodialysis access includes a needle access port having a housing defining a septum, wherein the septum is configured for needle access. The device also includes a valve having an open position and a closed position, wherein the valve is further configured to be anastomosed to a large-diameter blood vessel. An elongate tube is included in the device and has a wall defining a first and a second elongate lumen. Both the first and the second elongate lumens extend from a first end of the elongate tube to a second end of the elongate tube, and a first end of the tube is coupled to the needle access port and a second end of the tube is coupled to the valve.

In accordance with another aspect of the present invention the housing of the needle access port can include three septa. A first needle access port septum is configured for a needle to draw blood from the large-diameter blood vessel, a second needle access port septum is configured for a second needle to return blood to the large-diameter blood vessel, and the third needle access port septum is configured for a third needle to activate the valve. The second lumen of the elongate tubing can be configured to branch off from the first lumen of the elongate tubing such that it can be connected to the third needle access port. The housing of the needle access port is selected from the group of one of a biocompatible polymer or titanium. The housing of the needle access port can include a covering formed from silicone and can be configured such that a needle is insertable through the covering.

According to another aspect of the present invention, the housing of the needle access port further defines a nozzle allowing the elongate tube to be attached and detached from the needle access port. The elongate tube can include a locking mechanism positioned at the first end of the elongate tube for coupling the tube to the nozzle. Additionally, the first lumen of the elongate tube has a larger diameter than the second lumen of the elongate tube. The elongate tubing can be formed from a biocompatible material. The valve further can include a flap wherein the flap is sutured to the large-diameter vessel such that the valve is flush with a wall of the large-diameter blood vessel. The flap can be one of the group of Dacron and ePTFE.

According to yet another aspect of the present invention, the valve can take the form of a bi-stable valve contained in a housing and coupled to the flap, such that when the valve is in the closed position it is flush with the wall of the large-diameter blood vessel. When the bi-stable valve is in the closed position, the first and second lumen of the elongate tube are in fluid communication with each other and are blocked from having fluid communication with a blood stream in the large-diameter blood vessel and wherein when the bi-stable valve is in the open position the first lumen is in fluid communication with the blood stream and the second lumen is blocked from fluid communication with the blood stream in the large-diameter blood vessel. The bi-stable valve can include a durable biocompatible material able to withstand frequent movement and blood flow and able to be flipped from the closed position to the open position by pulling fluid from the third needle access port septum with a needle creating a negative pressure to flip the valve to the open position. The valve can be made from a durable biocompatible material able to withstand frequent movement and blood flow and able to be flipped from the open position to the closed position by injecting fluid into the third needle access port septum with a needle creating a negative pressure to flip the valve to the closed position. The third needle access port septum is positioned below one selected from the group consisting of the first and the second septum and separated from the one of the first and second septum by a silicone layer. In addition, the third needle access port septum can be accessible via an inner cannula which can be removed from a larger outer cannula disposed in the one of the first and second septum. The device can further include that the first and second access port septa, the first and second valves and the elongate tube are configured such that a cleaning fluid can be injected into the device though the first access port septum and removed from the second access port septum, when both the first and second valve are in the closed position. Additionally, a housing of the device can include ridges positioned along the outer surface and the housing can define a fluted opening for directing the flow of blood.

In accordance with still another aspect of the present invention a method of providing hemodialysis using a hemodialysis device includes anastomosing a first valve and a second valve to a large-diameter blood vessel such that the valves are in fluid communication with a flow of blood through the large-diameter blood vessel. The method also include coupling the first valve to a first septum of a needle access port using a first elongate tube having a first lumen and a second lumen and wherein the first lumen is attached to a first nozzle of the valve and the second lumen is attached to a second nozzle of the valve. Additionally, the method includes coupling the second valve to a second septum of the needle access port using a second elongate tube having a first lumen and a second lumen wherein the first lumen is attached to a first nozzle of the valve and the second lumen is attached to a second nozzle of the valve. The method can further include using a needle to pull fluid from a third septum of the needle access port to create a negative pressure to flip open the first and second valves such that the hemodialysis can be performed. Also, the method can include using a needle to inject fluid into the third septum of the needle access port to create a pressure to push the first and second valves closed.

In accordance with another aspect of the present invention, the method can include accessing the third septum of the needle access port by an inner cannula that can be removed from a larger outer cannula that remains in one of the first or second septum. The method can also include cleaning the hemodialysis device by closing the first valve and the second valve and injecting cleaning fluid through the second lumen of the first elongate tube coupled to the first valve and extracting the cleaning fluid from the second lumen of the second elongate tube coupled to the second valve. The method can further include anastomosing the first and second valve to the large-diameter blood vessel using a valve skirt.

### BRIEF DESCRIPTION OF THE DRAWINGS

The accompanying drawings provide visual representations which will be used to more fully describe the representative embodiments disclosed herein and can be used by those skilled in the art to better understand them and their inherent advantages. In these drawings, like reference numerals identify corresponding elements and:

FIG. 1 illustrates a perspective view of a hemodialysis device according to an embodiment of the invention.

FIG. 2 illustrates a top down view of a triple-port according to an embodiment of the invention.

FIG. 3 illustrates a partially sectional view of a housing and a valve disposed within the housing, according to an embodiment of the invention.

FIG. 4 illustrates a first side view of an outer housing according to an embodiment of the invention.

FIG. 5 illustrates a second side view of the outer housing according to the embodiment of the invention illustrated in FIG. 4.

FIG. 6 illustrates a top down view of the outer housing according to the embodiment of the invention illustrated in FIGS. 4 and 5.

FIG. 7 illustrates a perspective view of an inner housing according to the embodiment of the invention illustrated in FIGS. 4-6.

FIG. 8 illustrates a perspective view of a lower housing according to an embodiment of the invention.

FIG. 9 illustrates a perspective view of a valve according to an embodiment of the invention.

FIG. 10 illustrates a partially sectional view of an open valve according to an embodiment of the invention.

FIG. 11 illustrates a partially sectional view of a closed valve according to an embodiment of the present invention.

FIG. 12 illustrates a hemodialysis device conducting dialysis according to an embodiment of the present invention.

FIG. 13 illustrates a hemodialysis device configured for cleaning according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides a device and method for hemodialysis including a needle access port having a housing defining a septum, wherein the septum is configured for needle access. Generally, the needle access port will have three septa for communication with valves having an open position and a closed position. The valves are further configured to be anastomosed to a large-diameter blood vessel such that the valves are in fluid communication with a flow of blood through the large-diameter blood vessel. The device also includes an elongate tube having a first and a second elongate lumen, wherein a first end of the tube is coupled to the needle access port and a second end of the tube is coupled to the valve. The device can be cleaned by injecting a cleaning fluid into a first septum of the needle access port and extracting it though the second septum of the needle access port.

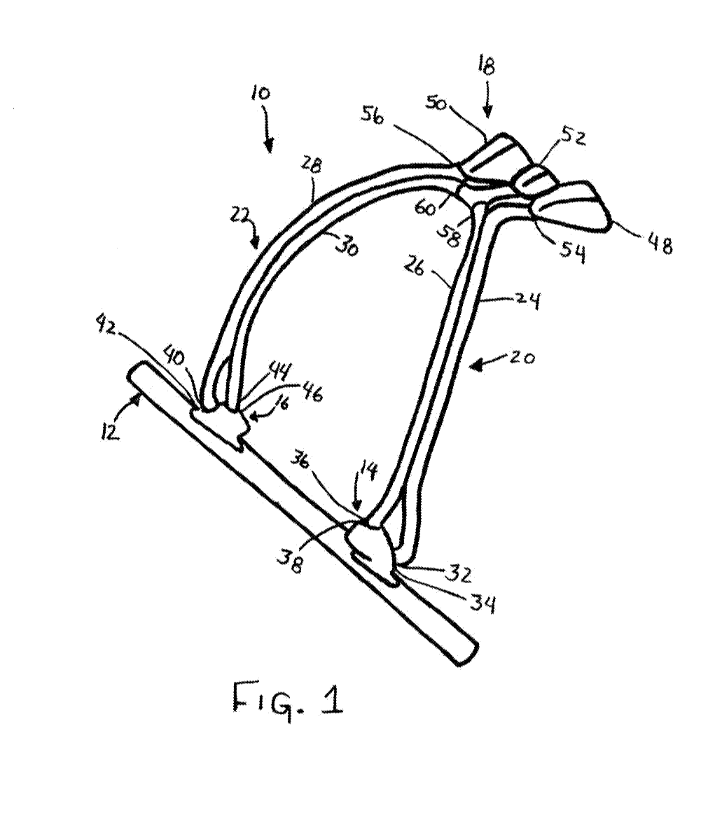

FIG. 1 illustrates a perspective view of a hemodialysis device according to an embodiment of the invention. As illustrated in FIG. 1, the hemodialysis device 10 is shown anastomosed to a large-diameter vein 12 such as the femoral vein. The hemodialysis device 10 includes a first valve assembly 14 and a second valve assembly 16 which are both anastomosed to the vein 12. The device 10 also includes a needle-access port 18 that is coupled to the first valve assembly 14 with a first elongate inlet tube 20 and is coupled to the second valve assembly 16, via a second elongate outlet tube 22. The first elongate inlet tube 20 includes a first lumen 24 and a second lumen 26 and the second elongate outlet tube 22 also includes a first lumen 28 and a second lumen 30. A first end 32 of the first lumen 24 of the first elongate inlet tube 20 couples to a first nozzle 34 on the first valve assembly 14, and a first end 38 of the second lumen 26 couples to a second nozzle 36 on the first valve assembly 14. Similarly, a first end 40 of the first lumen 28 of the second elongate outlet tube 22 couples to a first nozzle 42 on the second valve assembly 16, and a first end 44 of the second lumen 30 couples to a second nozzle 46 on the second valve assembly 16. All of the tubing can be formed from flexible 3-6 mm diameter silicone or silicone-like tubes, with 0.5-1.5 mm wall thickness and include bioengineered collapse prevention. Alternately, the tubing can be formed from any other suitable material known to one of skill in the art.

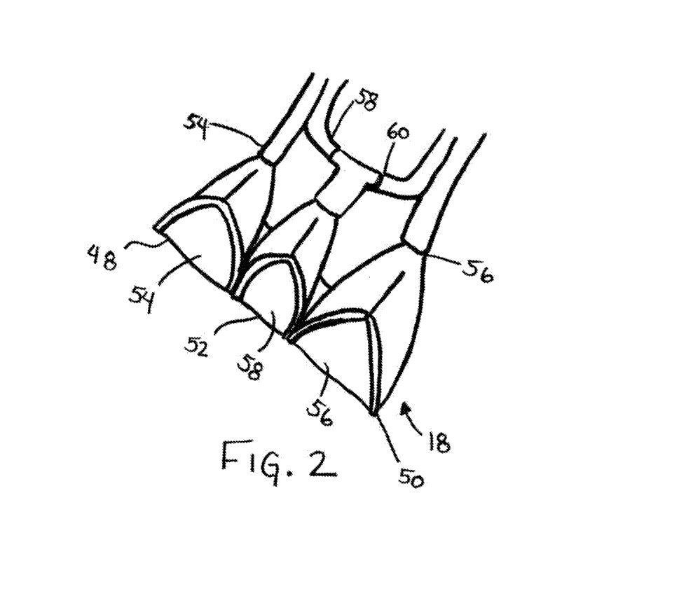

As illustrated in FIGS. 1 and 2, which illustrates a top down view of a triple-port according to an embodiment of the invention, the needle valve port 18 includes a first septum 48, a second septum 50, and a third septum 52. The first septum 48 couples to a second end 54 of the first lumen 24 of the first elongate inlet tube 20, and the second septum 50 is coupled to a second end 56 of the first lumen 28 of the second elongate outlet tube 22. Therefore, the first septum 48 and the second septum 50 are in fluid communication with a stream of blood traveling through the large-diameter vein 12. In addition, the third septum 52 is coupled to second ends 58, 60 of the second lumens 26, 30 of the first and second elongate inlet and outlet tubes 20, 22. The joined region of second lumens 26 and 30 can also be referred to as a connecting bridge. Therefore, the third septum 52 is in fluid communication with the first and second valve assemblies 14, 16 and, in turn, is in fluid communication with the first lumens 24, 28 of the first and second elongate inlet and outlet tubes 20, 22. The third septum 52 can be used to open and close valve assemblies 14 and 16 using fluid pressure, and can also be used as the hub for injecting and draining a biocompatible cleaning solution from the system.

Further, as illustrated in FIGS. 1 and 2 septa 48 and 50 are larger in size than the third septum 52. The first and second septa 48 and 50 are configured to be tolerant to multiple needle sticks with 14-17 gauge hemodialysis needles. These septa 48 and 50 are large to provide durability for large needle sticks. However, the first and second septa 48 and 50 can also be optimized to have smooth, angled surfaces for patient comfort. The third septum 52 is configured for controlling the valve assemblies 14 and 16. Saline can be pushed into or drained from the third septum 52 through a 22-25 gauge needle. The housing of the needle port 18 can be formed from titanium or a biocompatible polymer and puncture beds 54, 56, and 58 can be formed from a thick silicone or silicone-like layers that seal off around the needle after dialysis is completed. While this embodiment has been described in detail above, it should be known that any configuration known to one of ordinary skill in the art could be used to achieve these results and the above description should not be considered limiting.

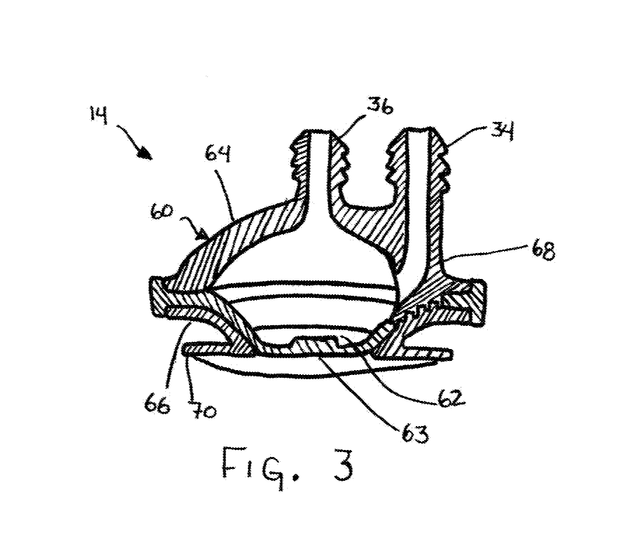

FIG. 3 illustrates a partially sectional view of a valve assembly disposed within the housing, according to an embodiment of the invention. As illustrated in FIG. 3, the valve assemblies 14 and 16 have the same structure that will be described in further detail, with respect to valve assembly 14, below. The valve assembly 14 includes a housing 60 surrounding a valve 62. The housing 60 can include a two-piece construction having a top-portion 64 and a bottom portion 66. The housing is illustrated as having a generally spherical shape, but any other suitable shape could be used. The top-portion 64 and the bottom-portion 66 can be coupled together, such that the valve 62 is sandwiched between them. The top-portion 64 of the housing 60 includes two nozzles, such as 34 and 36, as illustrated with respect to the first valve assembly 14, in FIG. 1. The housing can be formed from a semi-still silicone or other soft and semi-stiff material and the valve can be formed from durable silicone or a silicone-like material with an ePTFE coating at the surface 63 of the valve that is in contact with a flow of blood through the large-diameter vein. It should be noted that any suitable materials or coating that are known to one of ordinary skill in the art could also be used to construct the valve.

As illustrated in FIG. 3, the first nozzle 34 directs the flow of fluid out, as is the case with respect to the first valve assembly 14 and directs the flow of fluid in, as is the case with respect to the second valve assembly 16. The second nozzle 36 is incorporated into the housing 60 such that the valve assembly 14 can be placed in fluid communication with the third septum 52 of the needle port 18, such that the valve 62 can be opened and closed with fluid pressure and cleaning fluid can be introduced into the system. As illustrated in FIG. 3 the first nozzle 34 can include a fluted flow channel 68, which funnels fluid immediately to expose the valve 62 to reduced shear and in turn increases the stability of the valve 62. The nozzles 34 and 38 can be arranged in parallel as illustrated in FIG. 3 or perpendicularly, as illustrated in FIG. 1.

Further, as illustrated in FIG. 3, the bottom-portion 66 of the housing includes a flap or skirt 70 that can be formed from a Dacron, ePTFE, or any other flexible and easily sutured material known in the art. When implanted, a 1 cm incision is made to the vein and the skirt 70 is sutured to the edges of the incision. The skirt 70 is flexible, but pre-shaped to fit flush with the preferably 7-9 mm vein. When the valve 62 is in the closed position, it is tight against the lower housing, and flush with the anastomosed skirt, allowing for undisturbed blood-flow past the device when off-dialysis.

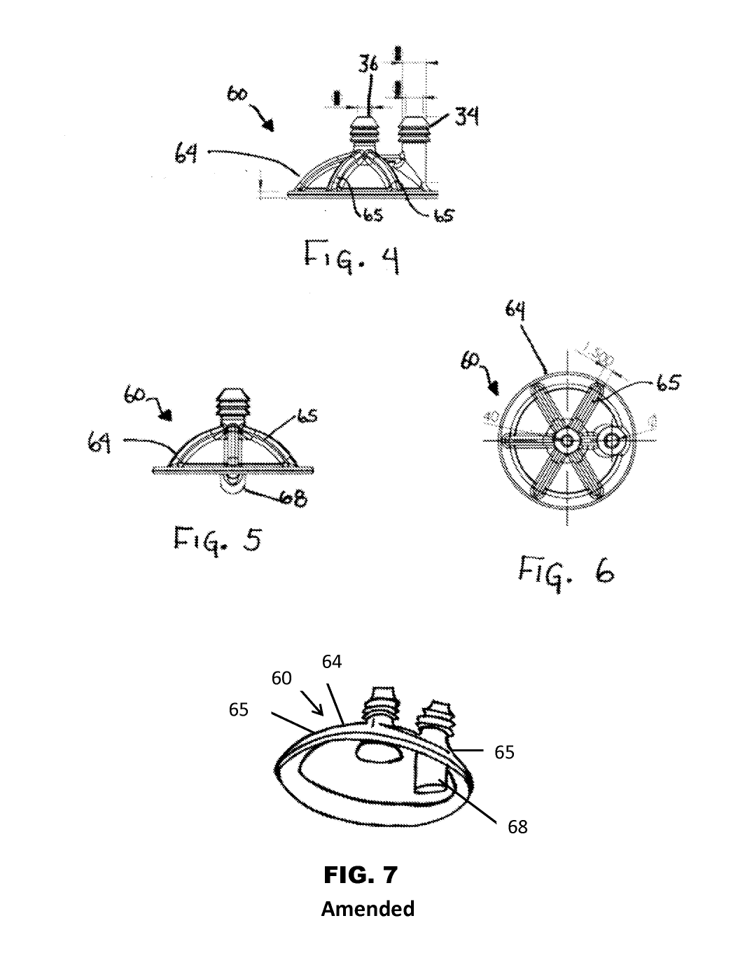

FIG. 4 illustrates a first side view of an outer housing according to an embodiment of the invention, and FIG. 5 illustrates a second side view of the outer housing according to the embodiment of the invention illustrated in FIG. 4. FIG. 6 illustrates a top down view of the outer housing according to the embodiment of the invention illustrated in FIGS. 4 and 5 and FIG. 7 illustrates a perspective view of an inner housing according to the embodiment of the invention illustrated in FIGS. 4-6. In the embodiment illustrated in FIGS. 4, 5, and 6, the housing 60 includes a top-portion 64 that includes nozzles 34 and 36. The top-portion 64 also preferably includes ridges 65 that enhance the ridgidity of the housing. FIGS. 5 and 7 also illustrate the structure of the flow channel 68, which directs the flow of fluid into and out of the valve assembly 14. The flow channel 68 is preferably fluted in shape to guide the direction of the flow of blood.

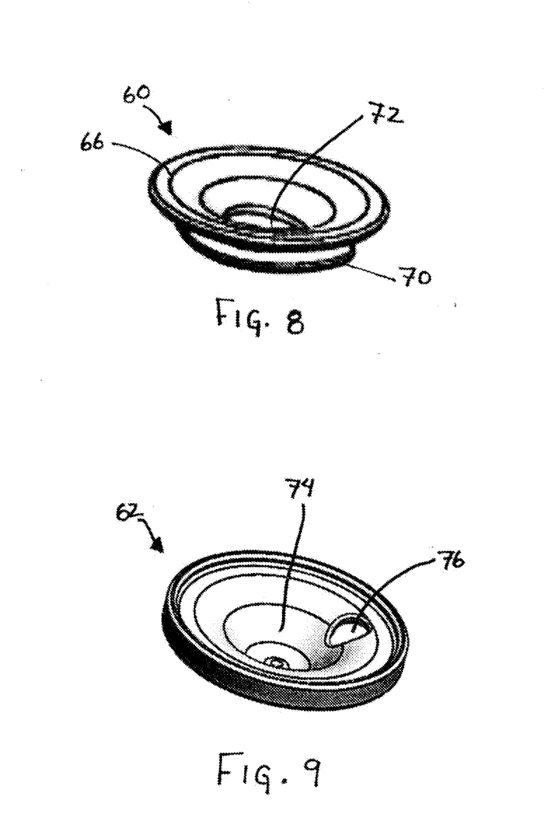

FIG. 8 illustrates a perspective view of a lower housing according to an embodiment of the invention. The bottom-portion 66 of housing 60 is illustrated in FIG. 8. The bottom-portion 66 includes the skirt 70 that is anastomosed to the large-diameter vein, as illustrated in FIG. 1. When the valve (not shown) is in the open position, fluid can flow through the opening 72 defined by the bottom-portion 66 of the housing 60. The bottom-portion 66 of the housing 60 couples with the top-portion (not shown), in order to sandwich the valve (not shown) in between the two portions of the housing 60. Therefore, both the bottom-portion 66 and the top-portion (not shown) are configured to be coupled, to form a fluid-tight structure surrounding the valve.

FIG. 9 illustrates a perspective view of a valve according to an embodiment of the invention. As described above, the valve 62 is configured to sit between the top-portion (not shown) of the housing 60 and the bottom-portion (not shown) of the housing 60. The valve 62 is further configured to have hang-down 74 that is flush with the skirt (not shown), when the valve 62 is in the closed position. The valve 62 also defines an opening 76, through which the flow channel (not shown) extends, when the valve 62 is in an open position. This helps to secure the valve in place between the top-portion (not shown) and the bottom-portion (not shown) of the housing (not shown) and also allows for the flow of blood through the valve and out of the valve assembly (not shown) through nozzle (not shown).

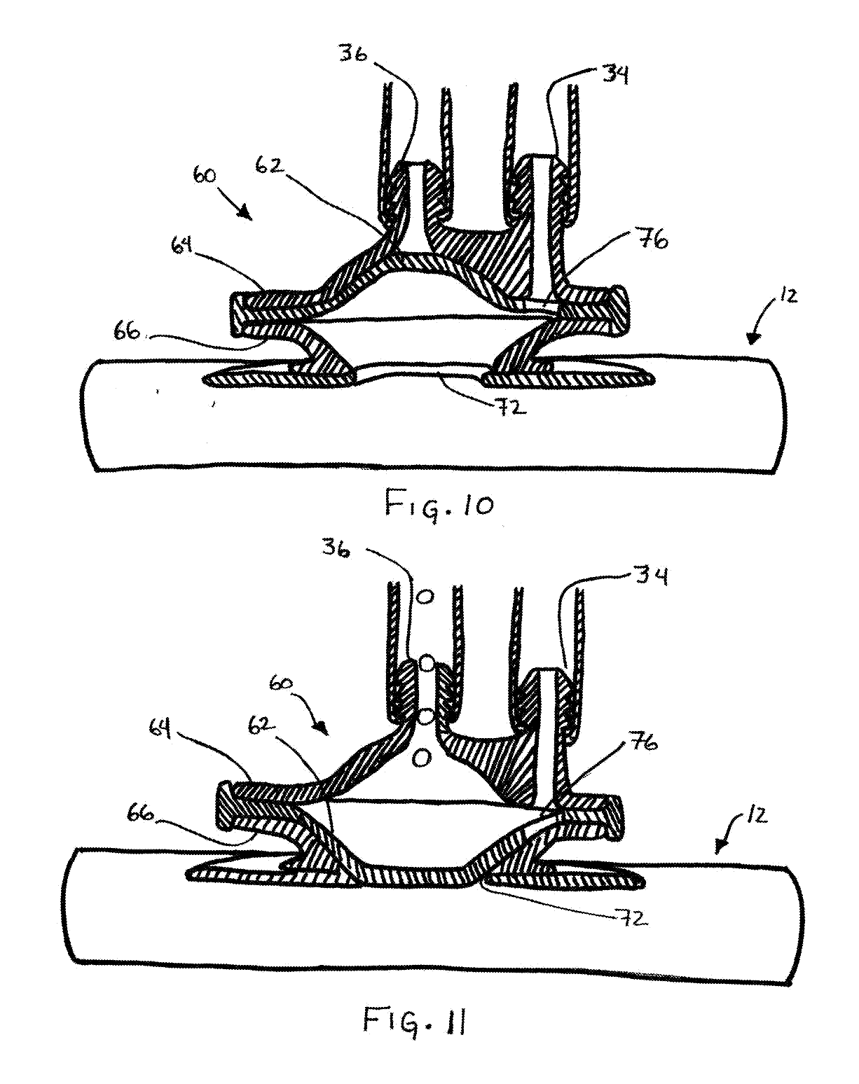

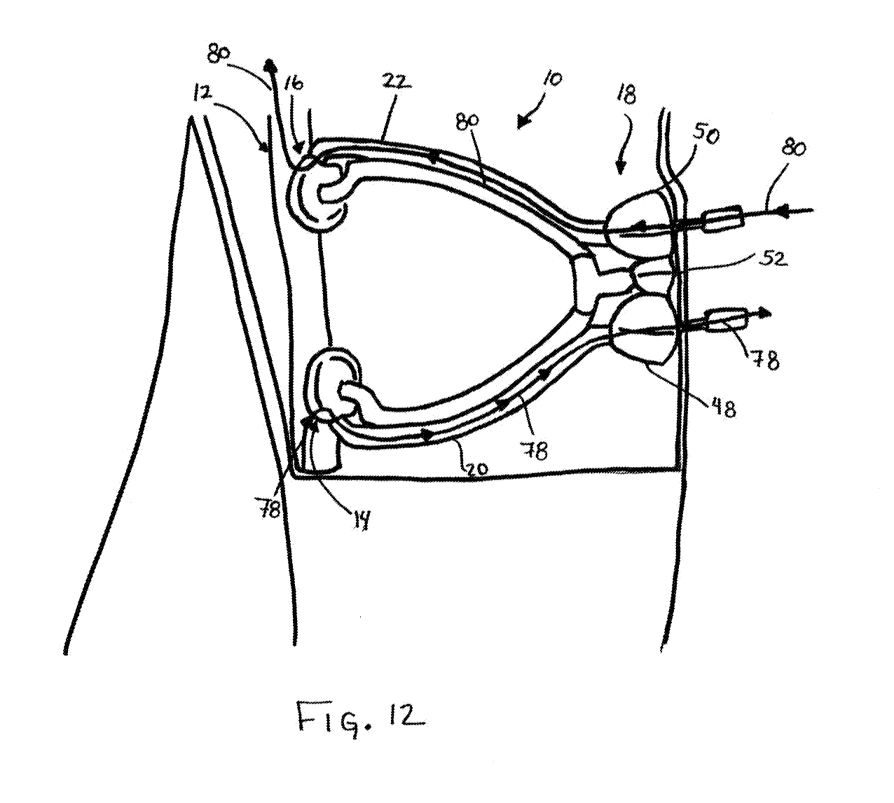

FIG. 10 illustrates a sectional view of an open valve according to an embodiment of the invention, and FIG. 11 illustrates a sectional view of a closed valve according to an embodiment of the present invention. As described above, when fluid is removed from the third septum of the needle port the valve 62 is opened, as illustrated in FIG. 10, and when fluid is injected the valve 62 is closed, as illustrated in FIG. 11. FIGS. 10 and 11 both show the valve 62 disposed within housing 60 of the valve assembly 14 or 16. As described with respect to FIG. 9, the valve 62 defines an opening 76 which, when open, as in FIG. 10, blood can flow through the opening 76 and through nozzle 34. In turn, when the valve 62 is closed, as in FIG. 11, the valve 62 is pressed against the bottom portion 66 of the housing 60, such that the opening 72 defined by the bottom portion of the housing is sealed against the flow of liquid between the valve assembly and the large-diameter vein 12.

FIG. 12 illustrates a hemodialysis device conducting dialysis according to an embodiment of the present invention. As illustrated in FIGS. 1 and 12 the valve assemblies 14 and 16 of the hemodialysis device 10 are anastomosed to the large-diameter vein 12. As illustrated by first flow direction arrow 78, shown in FIG. 12, blood flowing through the large-diameter vein 12 travels in through valve assembly 14, through the first lumen 24 of the first elongate inlet tube 20, and out through the first septum 48 of the needle port 18 for hemodialysis processing. After hemodialysis processing is complete, the blood flows back in as illustrated by second flow direction arrow 80. Flow direction arrow 80 follows a path in through the second septum 50 of the needle port 18 through the first lumen 28 of the second elongate outlet tube 22 into valve assembly 16 and back into the large-diameter vein 12.

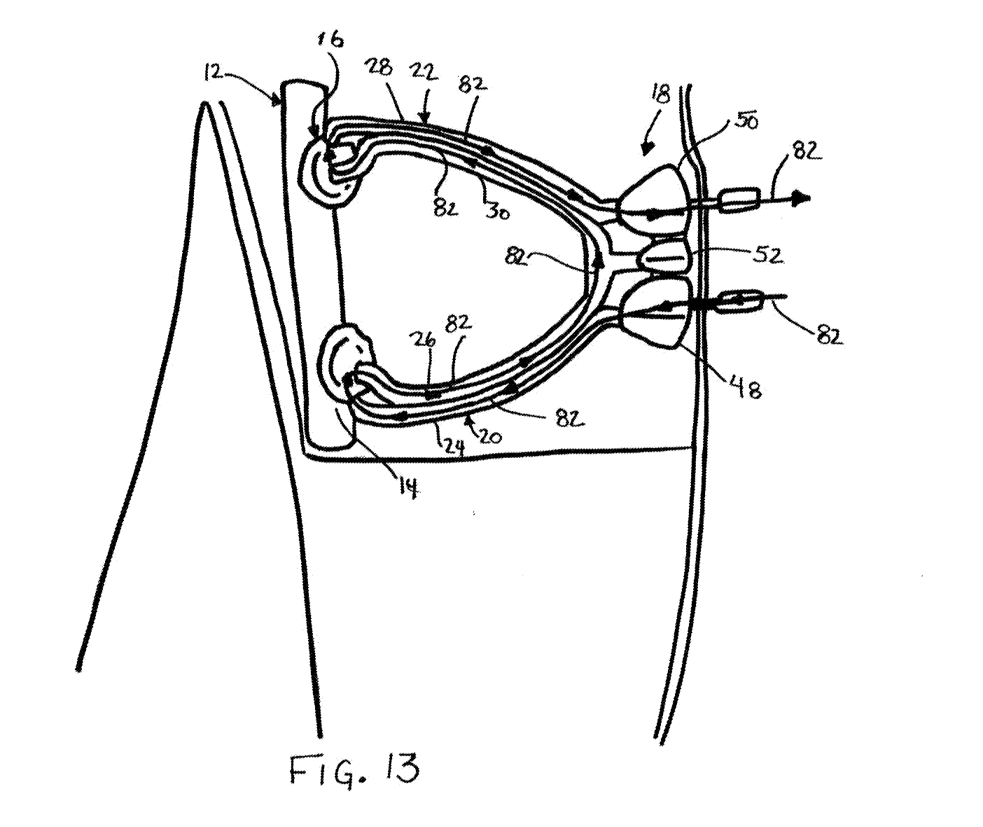

FIG. 13 illustrates a hemodialysis device configured for cleaning according to an embodiment of the present invention. During cleaning the valve assemblies 14 and 16 are closed to blood flow from the large-diameter vein 12, via the injection of fluid into the third septum 52 of the needle port 18. A cleaning solution can be injected into the device via the first septum 48 of the needle port 18. The cleaning solution follows a cleaning solution flow path 82 in through the first lumen 24 of the first elongate inlet tube 20 into closed valve assembly 14 into the second lumen 26 of the first elongate inlet tube 20 across bridge 84 and into the second lumen 30 of the second elongate outlet tube 22. The path 82 continues through the valve assembly 16 and into the first lumen 28 of the second elongate outlet tube 22 and then out through the second septum 50 of the needle port 18.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

Although the present invention has been described in connection with preferred embodiments thereof, it will be appreciated by those skilled in the art that additions, deletions, modifications, and substitutions not specifically described may be made without departing from the spirit and scope of the invention as defined in the appended claims.

What is claimed is:

1. A device for hemodialysis access comprising:
a needle access port having a housing defining a septum, wherein the septum is configured for needle access;
a valve having an open position and a closed position, wherein the valve is further configured to be anastomosed to a large-diameter blood vessel; and,
an elongate tube having a wall defining a first and a second elongate lumen and both the first and the second elongate lumens extending from a first end of the elongate tube to a second end of the elongate tube, wherein a first end of the tube is coupled to the needle access port and wherein a second end of the tube is coupled to the valve.

2. The device for hemodialysis access of claim 1, wherein the housing of the needle access port comprises three septa, wherein a first needle access port septum is configured for a needle to draw blood from the large-diameter blood vessel, a second needle access port septum is configured for a second needle to return blood to the large-diameter blood vessel, and the third needle access port septum is configured for a third needle to activate the valve.

3. The device for hemodialysis access of claim 2 wherein the second lumen of the elongate tubing is configured to branch off from the first lumen of the elongate tubing such that it can be connected to the third needle access port.

4. The device for hemodialysis access of claim 1 wherein the housing of the needle access port is selected from the group consisting of one of a biocompatible polymer or titanium.

5. The device for hemodialysis access of claim 1 wherein the housing of the needle access port comprises a covering formed from silicone and configured such that a needle is insertable through the covering.

6. The device for hemodialysis access of claim 1 wherein the housing of the needle access port further defines a nozzle allowing the elongate tube to be attached and detached from the needle access port.

7. The device for hemodialysis of claim 6 wherein the elongate tube further comprises a locking mechanism positioned at the first end of the elongate tube for coupling the tube to the nozzle.

8. The device for hemodialysis access of claim 1 wherein the first lumen of the elongate tube has a larger diameter than the second lumen of the elongate tube.

9. The device for hemodialysis access of claim 1 wherein the elongate tubing comprises a biocompatible material.

10. The device for hemodialysis access of claim 2 wherein the valve further comprises a flap wherein the flap is sutured to the large-diameter vessel such that the valve is flush with a wall of the large-diameter blood vessel.

11. The device for hemodialysis of claim 10 wherein a material for the flap is selected from one of the group of Dacron and ePTFE.

12. The device for hemodialysis of claim 10 wherein the valve further comprises a bi-stable valve contained in a housing and coupled to the flap, such that when the valve is in the closed position it is flush with the wall of the large-diameter blood vessel.

13. The device for hemodialysis of claim 12 wherein, when the bi-stable valve is in the closed position, the first and second lumen of the elongate tube are in fluid communication with each other and are blocked from having fluid communication with a blood stream in the large-diameter blood vessel and wherein when the bi-stable valve is in the open position the first lumen is in fluid communication with the blood stream and the second lumen is blocked from fluid communication with the blood stream in the large-diameter blood vessel.

14. The device for hemodialysis of claim 10 wherein the bi-stable valve comprises a durable biocompatible material able to withstand frequent movement and blood flow and able to be flipped from the closed position to the open position by pulling fluid from the third needle access port septum with a needle creating a negative pressure to flip the valve to the open position.

15. The bi-stable valve of claim 10 wherein the valve further comprises a durable biocompatible material able to withstand frequent movement and blood flow and able to be flipped from the open position to the closed position by injecting fluid into the third needle access port septum with a needle creating a negative pressure to flip the valve to the closed position.

16. The device of claim 2 wherein the third needle access port septum is positioned below one selected from the group consisting of the first and the second septum and separated from the one of the first and second septum by a silicone layer.

17. The device of claim 1 wherein the valve further comprises a housing having ridges along an outer surface of the housing.

18. The device of claim 1 wherein the valve further comprises a fluted opening to direct the flow of blood.

19. A method of providing hemodialysis using a hemodialysis device comprising:
anastomosing a first valve and a second valve to a large-diameter blood vessel such that the valves are in fluid communication with a flow of blood through the large-diameter blood vessel;
coupling the first valve to a first septum of a needle access port using a first elongate tube having a first lumen and a second lumen and wherein the first lumen is attached to a first nozzle of the valve and the second lumen is attached to a second nozzle of the valve;
coupling the second valve to a second septum of the needle access port using a second elongate tube having a first lumen and a second lumen wherein the first lumen is attached to a first nozzle of the valve and the second lumen is attached to a second nozzle of the valve;
using a needle to pull fluid from a third septum of the needle access port to create a negative pressure to flip open the first and second valves;
performing hemodialysis;
using a needle to inject fluid into the third septum of the needle access port to create a pressure to push the first and second valves closed.

20. The method of claim 19 further comprising cleaning the hemodialysis device by closing the first valve and the second valve and injecting cleaning fluid through the second lumen of the first elongate tube coupled to the first valve and extracting the cleaning fluid from the second lumen of the second elongate tube coupled to the second valve.
